(19) United States
(12) Patent Application Publication
(10) Pub. No.: US 2005/0186652 A1
(43) Pub. Date: Aug. 25, 2005

(54) Electrochemical detection

(76) Inventors: Luet L. Wong, Oxford (GB); Stephen Bell, Oxford (GB)

Correspondence Address: QUARLES & BRADY LLP, 411 E. WISCONSIN AVENUE, SUITE 2040, MILWAUKEE, WI, 53202-4497, US

(21) Appl. No.: 11/081,032

(22) Filed: Mar. 15, 2005

Related U.S. Application Data
(63) Continuation-in-part of application No. 10/505,341, filed on Nov. 8, 2004.

(30) Foreign Application Priority Data
Feb. 22, 2002 (GB) GB 0204232.3
Feb. 21, 2003 (WO) PCT/GB03/00751

(51) Int. Cl. C12Q 1/26

(52) U.S. Cl. 435/25

(57) ABSTRACT

A method for detecting the presence or absence of, or for determining the concentration of, a substrate in a sample is provided, wherein the method comprises
contacting a redox enzyme, NAD⁺ or NADP⁺, a reductase and a redox active agent with said sample; and measuring the quantity of reduced redox active agent produced by the reductase, by electrochemical means.

### FIELD OF THE INVENTION

This invention relates to assays for determining the amount of NADPH (nicotinamide adenine dinucleotide phosphate) or NADH (nicotinamide adenine dinucleotide) present in a sample. The assays may also be used to determine the activity or amount of a redox enzyme or its substrate.

### BACKGROUND OF THE INVENTION

NADH and NADPH are physiological substances which occur in all living cells including human cells. Both are byproducts of a large number of redox enzymes and in particular dehydrogenases. Many dehydrogenases employ NAD⁺ or NADP⁺ as a cofactor. As the substrate of the dehydrogenase is oxidised, NAD⁺ or NADP⁺ is simultaneously reduced to NADH or NADPH. This reduction involves the transfer of two electrons plus a proton (or hydride transfer) to the NAD⁺ or NADP⁺ to generate the NADH or NADPH. In the case of some dehydrogenases, such as glutamate dehydrogenase, the reaction can be driven in the reverse direction.

A large range of dehydrogenases exist and carry out a number of biologically important functions. Substrates for the dehydrogenases include biological important compounds such as amino acids, lactate, cholesterol and glycerol. It is important to be able to monitor the amount of these compounds and assays exist which employ dehydrogenases to measure the amount of such metabolites in biological samples.

The assays typically measure the amount of NADH or NADPH produced as the dehydrogenase oxidises the substrate and hence allow the quantification of the substrate. These assays typically employ spectrophotometric methods to detect the amount of NADH or NADPH. However, such spectrophotometric assays are cumbersome and require skilled operators to carry them out. Additionally, problems or costs may be involved where the sample contains high amounts of a protein which absorbs at the wavelength being measured or structures such as blood cells which also interfere with the absorbance. This may mean extra processing steps have to be carried out.

Electrochemical methods have been attempted to try and measure the amount of NADH or NADPH. However, the only methods developed so far typically use organic dyes such as Mendola blue and methyl-viologen or diaphorose as mediators and these are unstable and are frequently toxic. In addition, the electrochemical methods typically use electron acceptors that can only accept a single electron at a time from NADH or NADPH or which are too slow meaning that too small a current is produced or the current produced is not proportional to the amount of substrate present and hence quantitative results cannot be obtained.

### SUMMARY OF THE INVENTION

The present invention is based on the finding that a class of redox proteins known as reductases can simultaneously accept two electrons from NADH or NADPH and rapidly transfer them to a small, redox active molecule. The re-oxidation of the redox active molecule can then be detected, typically by using electrochemical methods. The fast rate of electron transfer reduction of the redox active small molecule complex by the reductase means that an effective, catalytic current can be generated which is limited by, and can be linear to, the substrate concentration. Thus results can be obtained which are truly quantitative. The method also allows measurement over time. Therefore NADH or NADPH can be effectively quantified electrochemically without the need for toxic, unstable organic dyes. The assay is also far simpler to carry out than those in the art, meaning less experience is required to carry it out and reducing its expense.

Accordingly, the present invention provides a method for detecting the presence or absence of, or for determining the concentration of, NADH or NADPH in a sample comprising;
adding a reductase and a redox active agent to said sample; and
measuring the quantity of reduced redox active agent produced by the reductase.

In particular, the invention provides a method for detecting the presence or absence of, or for determining the concentration of, NADH or NADPH in a sample comprising;
contacting a reductase and a redox active agent with said sample; and
measuring the quantity of reduced redox active agent produced by the reductase, by electrochemical means.

The invention further provides an electrochemical cell comprising:
sample holding means;
a source of reductase;
a redox active agent; and
means for detecting and/or quantifying any current generated.

The invention also provides a kit comprising:
a reductase;
a redox active agent; and
a means for detecting the reduction of the redox active agent.

The present invention also provides an electrochemical method for detecting and/or quantifying the amount of a substrate in a sample, the method comprising
contacting a redox enzyme, NAD⁺ or NADP⁺, a reductase and a redox active agent with said sample, wherein the redox enzyme is capable of oxidising or reducing the substrate using NAD⁺, or NADP⁺ as a cofactor; and
measuring the quantity of reduced redox active agent produced by electrochemical means.

The present invention also provides an electrochemical method for detecting and/or quantifying the amount of a redox enzyme in a sample, the method comprising
contacting a substrate, NAD⁺ or NADP⁺, a reductase and a redox active agent with the sample, wherein the redox enzyme is capable of oxidising or reducing the substrate using NAD⁺ or NADP⁺ as a cofactor; and
measuring the quantity of reduced redox active agent produced by electrochemical means.

Also provided is an electrochemical method for detecting and/or quantifying the amount of a target analyte in a sample, the method comprising
reacting the target analyte with an enzyme to produce a substrate, or where the target analyte is an enzyme, reacting the target analyte with a precursor compound to produce a substrate,
contacting the thus produced substrate with a redox enzyme, NAD⁺ or NADP⁺, a reductase and a redox active agent, wherein the redox enzyme is capable of oxidising or reducing the substrate using NAD⁺ or NADP⁺ as a cofactor; and
measuring the quantity of reduced redox active agent produced by electrochemical means.

The invention also provides an electrochemical detection sensor comprising an electrode system having a working electrode, a reference electrode and optionally a counter electrode and a buffered mixture of (i) a dehydrogenase enzyme for converting a target analyte to a product, (ii) NAD⁺ or NADP⁺, (iii) a NADH or NADPH reductase and (iv) a redox active agent.

The invention also provides a method of operating such a sensor, the method comprising
contacting a liquid sample containing a target analyte with the mixture of enzymes and redox agent such that the target analyte is oxidised, producing NADH or NADPH which reduces the reductase, and the reductase in turn reduces the redox agent which is then oxidised at the working electrode; and
measuring the current produced.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

General Scheme for Assay

The general scheme for the assay system of the invention is as follows:

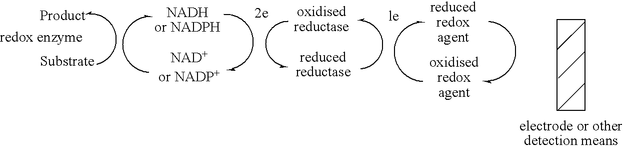

Although most commonly the assay scheme is as indicated above, the system can effectively be run in reverse, with electrons and a proton being transferred from the reductase to NAD⁺ or NADP⁺ to generate NADH or NADPH respectively. In this embodiment, the quantity of oxidised redox agent is monitored, for example by electron transfer to the electrode.

The assay can be used to detect and/or quantify the amount of the:
redox enzyme;
substrate; or
NADH, NADPH, NAD⁺, or NADP⁺ or an analog of any of these present in the biological sample. In particular, a direct measure of the amount of NADH or NADPH present or generated in the sample, can be correlated to the amount of substrate present in a sample, or amount of redox enzyme. In this embodiment, it is particularly useful to plot the generation of NADH/NADPH through the electrode via reductase and redox active agent, over time to elucidate the activity and thus presence and amount of the redox enzyme present.

The assay system can also involve additional steps. For example, additional stages may be involved after the reductase or before the redox enzyme indicated above. Such additional steps may also involve the transfer of electrons and will typically be enzyme catalysed. Where there are additional steps before the redox enzyme which coverts NAD⁺ or NADP⁺ to NADH or NADPH or vice versa the purpose of the assay may be to measure the amount of a target analyte which is an enzyme or compound upstream of the redox enzyme. Thus, for example, the method may involve reacting a target enzyme with a precursor compound to produce a substrate which is reacted in accordance with the above assay. Alternatively, the method may involve reacting a target compound with an enzyme to produce the substrate which is reacted in accordance with the above assay.

In one embodiment, the upstream events involve the release of a bound substance which can then be detected. Typically, the bound substance is released enzymatically. For example the bound substance may be cholesterol and the enzyme catalysing its release may be a lipase. Other substances or cofactors, such as a detergent, may also be present. Once released the substance, such as cholesterol, is then a substrate for the redox enzyme. Such assays are used to detect the presence or absence of, or to quantify the amount of, either the bound substance such as cholesterol or the enzyme catalaysing its release such as lipase. Preferably the assay is used to detect and/or quantify the bound substance.

In a preferred embodiment, the invention can be used to detect and/or quantify the amount of the substrate. In this embodiment, a sample containing the substrate is contacted with a redox enzyme, NAD⁺ or NADP⁺, a reductase and a redox active agent. Measurement of the quantity of reduced redox active agent, for example by measuring the current generated at an electrode, thus gives an indication as to the amount of substrate present in the sample. The assay can thus be carried out without separately measuring the amount of NADH or NADPH generated.

In this embodiment, a catalytic amount of NAD⁺ or NADP⁺ may be employed. The NAD⁺ or NADP⁺ which is converted to NADH or NADPH is, in accordance with the above assay, regenerated following reduction of the oxidised reductase. Due to this regeneration of NAD⁺ and NADP⁺, the assay can be carried out using a smaller number of molecules of NAD⁺ or NADP⁺ than the number of molecules of substrate to be detected. This embodiment of the invention is, for example, appropriate for detecting substrates such as cholesterol or glucose.

Analyte or Enzyme to be Measured

The redox enzyme responsible for the initial step in the assay can be any of a number of possible enzymes and the assay may be used to measure the amount of that enzyme or its substrate. The enzyme is matched with the substrate so that appropriate pairings are chosen such that the enzyme can oxidise or reduce the substrate using the nicotinamide adenine dinucleotide as a cofactor. Typically the enzyme oxidises the substrate and reduces NAD⁺, or NADP⁺. The enzyme may be a recombinant enzyme or one from a natural source.

Examples of possible enzymes which can be used or quantified include glycerol dehydrogenase, glucose dehydrogenase, glutamate dehydrogenase, cholesterol dehydrogenase, ethylene glycol dehydrogenase, lactate dehydrogenase, alcohol dehydrogenase, α-hydroxybutyrate dehydrogenase, isocitric dehydrogenase, sorbitol dehydrogenase, aspartate aminotransferase, and alanine anino-transferase. In a preferred embodiment of the invention the redox enzyme is a dehydrogenase and more preferably is glycerol dehydrogenase, glucose dehydrogenase glutamate dehydrogenase, ethylene glycol dehydrogenase, cholesterol dehydrogenase, lactate dehydrogenase, or alcohol dehydrogenase.

Any of a large number of compounds can be used as the substrate. The substrate may be a biologically important molecule such as urea, uric acid, ammonia, lactic acid, trigylyceride, LDL, salicylate, glucose, pyruvic acid, carbon dioxide, adenosine 5′-triphosphate, 2,3-diphosphoglycerate or cholesterol. Alternatively, the substrate may be a drug or a metabolite of a drug.

In some embodiments of the invention, the assays are used to quantify NADH, NADPH, NAD⁺, or NADP⁺ itself. For example, such assays may be used to study biological systems in which these compounds are involved. Alternatively, NADH and NADPH have been used to treat a number of conditions including such as atherosclerosis, AIDS, cancer, chronic fatigue syndrome, Parkinson's and Alzheimer's and the assay of the invention can be used to measure NADH or NADPH in biological samples from subjects to whom these compounds have been administered.

The amount of substrate, enzyme, NADH, NADPH, NAD⁺, or NADP⁺ employed in the assay typically depends on what is being measured and the system being used to measure the transfer of electrons through the reductase. One of the substrate, enzyme, NADH, NADPH, NAD⁺, or NADP⁺ is typically present in an unknown amount as it is the substance being quantified. Typically, where that compound is not the one being quantified, the amount of:
redox enzyme present is from 0 to 1000 μM, 1 to 100 μM, preferably from 5 to 75 μM, and even more preferably from 5 to 50 μM;
reductase present is from 0 to 1000 μM, 0.001 to 100 μM, preferably from 0.1 to 50 μM, and even more preferably from 1 to 10 μM;
NAD⁺, or NADP⁺ present is from 0.1 to 100 mM, preferably from 0.1 to 75 mM, and even more preferably from 0.1 to 50 mM; and/or
redox active agent is from 1 to 100 mM, preferably from 1 to 25 mM, and even more preferably from 1 to 10 mM.

Instead of using NADH, NADPH, NAD⁺, or NADP⁺, the assay may employ an analog of one of these compounds. Such analogs will typically be artificial or synthetic analogs chosen for particular characteristics such as their increased stability, e.g. to extremes of pH, or the way in which they interact with a particular redox enzyme, e.g. less inhibition by NADH. Examples of NAD⁺ analogues include APAD (Acetyl pyridine adenine dinucleotide), TNAD (Thio-NAD), AHD (acetyl pyridine hypoxanthine dinucleotide), NaAD (nicotinic acid adenine dinucleotide), NHD (nicotinamide hypoxanthine dinucleotide), NGD (nicotinamide guanine dinucleotide), PAAD (pyridine acetaldehyde adenine dinucleotide). Preferred examples of NAD⁺ analogues include TNAD, NaAD, NHD, NGD and PAAD, in particular TNAD, NHD and NGD. The equivalent NADP⁺ (and NADH or NADPH) analogues are also commercially available or can be prepared by those skilled in the art.

Reductases

The use of a reductase in the assay of the invention allows the rapid transfer of electrons either from the NADH or NADPH to the redox active agent or vice versa. The reductase typically receives two electrons from the NADH or NADPH and it is this acceptance of two electrons simultaneously followed by their rapid transfer individually to the redox active agent which allows a significant current to be generated, the size of which is directly proportional to the amount of substrate present and hence allowing meaningful, accurate results to be obtained.

The reductase employed may be any reductase capable of transferring the electrons rapidly enough to allow a detectable current to be generated. Preferred reductases include cytochrome P450 reductases. In particular, preferred reductases include the putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductases. Where a nitrate reductase is employed preferably corn nitrate reductase is used. Particularly preferred reductases for use in the invention are putidaredoxin reductase and flavin domain of P450_{BM-3} as these are capable of particularly rapid electron transfer and hence allow optimal results to be obtained. The reductase may alternatively be diaphorase, or a reductase other than diaphorase may be used.

The reductase may be a recombinant protein or a naturally occurring protein which has been purified or isolated. The reductase may have been mutated to improve its performance such as to optimise the speed at which it carries out the electron transfer or its substrate specificity. The amount of reductase employed will depend on the exact nature of what is measured and the particular details of the assay but typically, the reductase will be present at a concentration of from 0 to 1000 μM, 0.001 to 100 μM, preferably from 0.01 to 50 μM, more preferably from 0.1 to 25 μM, and even more preferably from 1 to 10 μM.

For putidaredoxin reductase, typically the amount of enzyme employed will be from 100 to 10,000 ca, preferably from 1000 to 5000 ca, more preferably from 2000 to 4000 ca and even more preferably will be 3000 ca. The ca is the unit of activity of putidaredoxin reductase in mediating the oxidation of NADH by ferricyanide and is defined as 1 μmole of NADH oxidised per mg reductase per minute.

The general nature of reduction of small molecules by the reductase means the redox potential at which the small molecule mediator is detected can be tuned by standard methods of coordination chemistry so as to avoid interference from redox active molecules found in naturally occurring biological fluids.

Redox Active Agent

A redox active agent is employed to accept the electrons from the reductase. Typically, the redox active agent will accept the electrons one at a time. The redox agent may be a redox active molecule or inorganic complex. The agent may be a naturally occurring electron acceptor such as a protein or may be a synthetic molecule. The agent will typically have at least two oxidation states to allow it to accept electrons from the reductase.

Preferably, the redox agent is an inorganic complex. The agent may comprise a metallic ion and will preferably will have at least two valencies. In particular, the agent may comprise a transition metal ion and preferred transition metal ions include those of cobalt, copper, iron, chromium, manganese, nickel, or ruthenium. The redox agent may be charged, for example it may be cationic or alternatively anionic. An example of a suitable cationic agent is a ruthenium complex such as Ru(NH₃)₆³⁺, an example of a suitable anionic agent is a ferricyanide complex such as Fe(CN)₆³⁻. The reaction scheme for each respectively is: 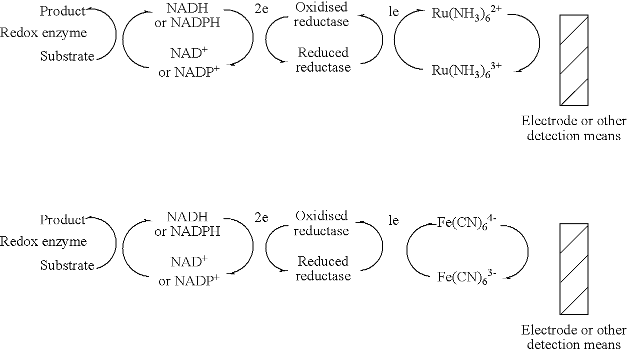
The redox active agent may be hydrophobic, for example a ferrocenium complex may be used, or may be hydrophillic. Example of complexes which may be used include Cu(EDTA)²⁻, Fe(CN)₆³⁻; Fe(CN)₅(O₂CR)³⁻, Fe(CN)₄(oxalate)³⁻, Ru(NH₃)₆³⁺ and chelating amine ligand derivatives thereof (such as ethylenediamine), Ru(NH₃)₅(py)³⁺, ferrocenium and derivatives thereof with one or more of groups such as —NH₂, —NHR, —NHC(O)R, and —CO₂H substituted into one or both of the two cyclopentadienyl rings. Preferably the inorganic complex is Fe(CN)₆³⁻, Ru(NH₃)₆³⁺, or ferrocenium monocarboxylic acid (FMCA).

The redox active agent interacts with the reductase and hence particular combinations of redox active agent and reductase may provide optimal results in terms of the rate of electron transfer or the charge produced or other desirable characteristics. For example, the reductase may have particular amino acids near its active site which mean that a hydrophobic redox agent is preferred or alternatively the region may contain positive or negatively charged residues or polar groups and hence an anionic or cationic redox agent may be more suitable. The particular pair of enzyme and redox agent is chosen to complement each other and hence to provide optimal results. Preferred pairings include Fe(CN)₆³⁻ or Ru(NH₃)₆³⁺ with putidaredoxin reductase or the flavin domain of P450_{BM-3} and either Fe(CN)₆³⁻ or Ru(NH₃)₆³⁺ with spinach ferredoxin reductase. The choice of redox agent may be selected based on the means used to detect the charge produced such as the electrode used.

The concentration of redox reagent used may vary depending on what is being measured and the other components in the assay, but typically is in the range of 0.01 to 500 mM, preferably from 0.1 to 100 mM, more preferably from 1 to 50 mM, and even more preferably from 5 to 25 mM.

In some embodiments, the methods of the present invention do not encompass the use of diaphorase as the redox active agent or organic dyes such as Mendola blue and methyl-viologen.

Detection Methods

The transfer of electrons to the redox agent to the redox agent is detected. This is preferably done by electrochemical means, although if desired NADH and NADPH concentrations can be measured at a wavelength of 340 nm or the Fe(CN)₆³⁻ concentration at 420 nm.

Typically, the redox agent transfers electrons to an electrode generating a current. The current generated is typically proportional to the amount of substrate present in the sample and hence the amount of the substrate can be determined.

A typical sensor system based on electrochemical detection which may be used in the invention comprises a working electrode, for example constructed from carbon paste, platinum, gold or some such suitable conducting material, a reference electrode which is typically the AgCl/Ag electrode, and optionally a counter electrode. All electrodes are fabricated by techniques such as screen printing which are well known to those familiar with the art. A buffered solution of the dehydrogenase enzyme for converting the target analyte substrate to its product(s), NAD⁺ or NADP⁺, a NADH or NADPH reductase, and the redox agent such as ferricyanide or Ru(NH₃)₆³⁺, can be deposited onto the electrode system and dried.

The whole or part of the sensor system may be covered by a membrane or a number of membranes or filters for example, to separate red cells from a blood sample or to ensure that undesired substances or cells do not enter the sensor.

When a liquid sample such as blood is applied, the liquid typically enters the sensor compartment and redissolves the mixture of enzymes and redox agent. The substrate (which is in some cases the target analyte, or which may be a compound produced by reaction of the target analyte with an enzyme or with a precursor compound) is then oxidised, producing NADH or NADPH which rapidly reduces the reductase. The reductase in turn rapidly reduces the redox agent such as Ru(NH₃)₆³⁺ to a reduced form, in this case Ru(NH₃)₆²⁺, which is then oxidised by the working electrode set at the appropriate potential. The current produced will be proportional to the concentration of the target analyte.

In another embodiment of the invention the working electrode may be located away from the dehydrogenase/NAD⁺/reductase/redox agent mixture to minimise fouling of the working electrode and thus improve sensor performance.

By using a dehydrogenase with the appropriate substrate specificity and altering the reaction conditions, for example by including suitable chemical and biological components such as lipids, detergents, surfactants or other proteins, a rapid sensor may be constructed. Additionally another enzyme system can be used to convert a target analtye into a substance which is oxidised by the dehydrogenase and then detected by the present electrochemical method. For example a sensor to detect and or quantify triglycerides may be constructed where triglycerides are cleaved to glycerol and fatty acids by a suitable lipase and the glycerol may then be detected by glycerol dehydrogenase coupled to the reductase/redox agent system of the invention.

Samples

The assay of the invention can be used to monitor the presence of a particular analyte or enzyme in a wide array of samples. Typically, the samples analysed are liquid and will usually be an aqueous fluid. In particular, the sample may be a bodily fluid such as urine, serum, blood or amniotic fluid, cerebrospinal fluid (CSF) or alternatively may be a fluid such as milk, wine or juice or have been prepared from one of these. The sample may be culture medium from prokaryotic or eukaryotic cells and in particular from mammalian cells. The sample may be a foodstuff or drink or may be the product of an industrial process and the assay used to monitor the product of a compound or the incidence of byproducts or toxic compounds.

Control samples may also be analysed. These may lack one or more of the substrate, redox enzyme, reductase, redox agent, NADH, NADPH, NAD⁺, or NADP⁺. The controls may be obtained from healthy individuals in order to compare them to those of a patient. Reference samples may be used which contain a known amount of the substrate or enzyme to be measured and hence allow calibration of the apparatus and/or standard curves to be drawn up to be compared against the results for the unknown samples. The sample may be serially diluted and several dilutions analysed as the concentration of the substrate or enzyme to be measured might be too high to be effectively measured in undiluted form.

The method of the invention can be used to diagnose or assess the progression of various diseases or to determine the susceptibility of an individual to such diseases. Typically, the method is used to detect the presence or absence of, or to quantify the amount of a compound or enzyme associated with a disease state. For example, the method may be use to detect or quantify the amount of glucose in urine or blood from a diabetic or alternatively to measure the level of cholesterol or particular enzymes in a patient suffering from heart disease. The method may also be used to measure a compound or enzyme to determine when a drug or therapy needs to be administered to a patient or to measure how much of a drug remains or reached its target once administered. The method can also be used to detect and quantify drugs of abuse in samples from individuals.

The methods of the invention can be used to monitor lipid levels such as cholesterol or triglycleride levels and in particular may be used to quantify the levels of lipids in the blood or plasma. This may allow detection of individuals at risk of conditions associated with abnormal lipid levels such as those associated with elevated levels of a lipid or lipid deficiency. It may also allow monitoring of the progress of treatment of such a condition to assess whether lipid levels have returned to normal. Conditions include atherosclerosis, stroke, cardiovascular disease and heart attack as well as lipid deficiencies resulting from inadequate diet or metabolic defects.

The methods of the invention can also be used to monitor other metabolic defects such as those involved, for example, in amino acid metabolism. Such conditions include phenylketonuria (PKU) which may be monitored or detected by using phenyl alanine dehydrogenase as the redox enzyme in the system of the invention.

Kits

The invention also provides kits for carrying out the methods of the invention. Typically, the kit will comprise:
a reductase;
a redox active agent; and
a means for detecting the reduction of the redox active agent.
   The kit may also comprise a redox enzyme, preferably a dehydrogenase, and/or the substrate for the redox enzyme. The redox enzyme, the substrate, reductase and redox active agent may be any of those discussed above.

The means for detecting the reduction of the redox agent may be an electrochemical means, such as an electrochemical cell or its constituent parts, or alternatively, but less preferably, may be spectrophotometric means.

The kit may include means for detecting and quantify current and optionally a computer system for analysing and displaying the results. The kit may also include various packing and suitable instructions detailing how to use the kit and how it should be stored.

### EXAMPLES

The following Examples further illustrate the present invention.

### Example 1

### Spectrophotometic Analysis for Putidaredoxin Reductase

Putidaredoxin reductase is specific to NADH. The oxidation of NADH by Fe(CN)₆³⁻ catalysed by putidaredoxin reductase was demonstrated by a spectrophotometric assay.

1.5 ml of an incubation mixture containing 50 mM phosphate buffer, pH 7.4, 0.1 nM putidaredoxin reductase, and 1 mM K₃Fe(CN)₆ was added to cuvettes. A spectrophotometer was used to measure the absorbance at 340 nm and set to zero using a cuvette containing the incubation mixture alone.

NADH was then added to some of the cuvettes containing the incubation mixture as a 30 mM stock in 50 mM phosphate buffer, pH 7.4 to a final concentration of 300 μM (A₃₄₀ₙₘ ca, 1.8) and absorbance at 340 nm monitored. As shown by FIG. 1a, the absorbance due to NADH decreased steadily with time as it was oxidised to NAD⁺ by ferricyanide acting as an oxidising agent in a reaction mediated by putidaredoxin reductase which catalysed the transfer of electrons from one to the other. If either the reductase or ferricyanide was omitted no change in absorbance was seen (FIG. 1b). The reaction can also be monitored at the ferrocyanide absorption at 420 nm.

The Ru(NH₃)₆³⁺ reduction assay was carried out under identical conditions except that the putidaredoxin reductase concentration was 1 μM and the Ru(NH₃)₆³⁺ concentration was 2 mM. NADH was added to a final concentration of Ca. 300 μM (A₃₄₀ₘₘ=1.8) and the NADH absorption at 340 mm was monitored. As shown in FIG. 1c the NADH was oxidised in less than 2 minutes. When putidareodoxin reductase (PdR) was absent there was no detectable oxidation of NADH (FIG. 1d).

### Example 2

### Spectrophotometric Analysis for the P450_{BM-3} Falvin Domain

The flavin domain of P450_{BM-3} is specific for NADPH. The oxidation of NADPH by both Fe(CN)₆³⁻ and Ru(NH₃)³⁺ catalysed by the flavin domain of P450_{BM-3} was demonstrated by spectrophotometric assays.

1.5 ml of an incubation mixture containing 50 mM phosphate buffer, pH 7.4, 50 nM of the flavin domain of P450_{BM-3}, and either 1 mM K₃Fe(CN)₆ or 2 mM Ru(NH₃)₆³⁺ was added to cuvettes. A spectrophotometer was used to measure the absorbance changes at the relevant wavelength.

For the ferricyanide reduction assay, the reaction was monitored at the 420 nm absorptions of ferricyanide. NADPH was added to the sample cuvette as a 30 mM stock in 50 mM phosphate, pH 7.4 to a final concentration of ca. 500 μM (A₃₄₀ₙₘ=3.0) and the absorbance at 420 nm monitored. As shown in FIG. 2, all the ferricyanide was reduced to the ferrocyanide in less than 80 seconds.

For the Ru(NH₃)₆³⁺ reduction assay, the reaction was monitored at the NADPH absorptions at 340 nm. NADPH was added to a final concentration of ca. 300 μM (A₃₄₀ₙₘ=1.8). As shown in FIG. 3, all the NADPH was consumed in less than 90 seconds.

### Example 3

### Spectrophotometric Analysis for Spinach Ferredoxin Reductase

Spinach ferredoxin reductase is specific for NADPH. The oxidation of NADPH by both Fe(CN)₆³⁻ and Ru(NH₃)₆³⁺ catalysed by spinach ferredoxin reductase was demonstrated by spectrophotometric assays.

1.5 ml of an incubation mixture containing 50 mM phosphate buffer, pH 7.4, 0.05 units of spinach ferredoxin reductase, and either 1 mM K₃Fe(CN)₆ or 2 mM Ru(NH₃)₆³⁺ was added to cuvettes. A spectrophotometer was used to measure the absorbance changes at the relevant wavelength.

For the ferricyanide reduction assay, the reaction was monitored at the 420 nm absorptions of ferricyanide reduction assay, the reaction was monitored at the 420 nm absorptions of ferricyanide. NADPH was added to the sample cuvette as a 30 mM stock in 50 mM phosphate, pH 7.4 to a final concentration of ca; 500 μM (A₃₄₀ₙₘ=3.0) and the absorbance at 420 nm monitored. As shown in FIG. 4a, all the ferricyanide was reduced to the ferrocyanide in approximately 60 seconds. NADPH oxidation was much slower when either the reductase or ferricyanide was absent, see FIGS. 4b, 4c.

For the Ru(NH₃)₆³⁺ reduction assay, the reaction was monitored at the NADPH absorptions at 340 nm. NADPH was added to a final concentration of ca. 400 μM (A₃₄₀ₙₘ=2.5). As shown in FIG. 5a, all the NADPH was consumed in less than 90 seconds. NADPH oxidation was much slower when either the reductase or Ru(NH₃)₆³⁺ was absent, see FIGS. 5b, 5c.

### Example 4

### Electrochemical Analysis

A sensor system based on electrochemical detection is constructed using a working electrode, constructed from carbon paste, platinum, gold or some such suitable conducting material, a reference electrode which is a AgCl/Ag electrode, and optionally a counter electrode. The electrodes may be prepared using screen printing.

A buffered solution of the dehydrogenase enzyme for converting the target analyte substrate to its product(s), NAD⁺ or NADP⁺, a NADH or NADPH reductase, and ferricyanide or Ru(NH₃)₆³⁺ as a redox agent, can be deposited onto the electrode system and dried.

A liquid sample is applied to the system, the liquid enters the sensor compartment and redissolves the mixture of enzymes and redox agent. The target analyte is then oxidised, producing NADH or NADPH which rapidly reduces the reductase. The reductase may be putidaredoxin reductase where NADH is used or the flavin domain of P450_{BM-3} or the spinach ferredoxin reductase where NADPH is employed. The reductase in turn rapidly reduces the redox agent such as Ru(NH₃)₆³⁺ or Fe(CN)₆³⁻ to a reduced form, in other words to Ru(NH₃)₆²⁺ or Fe(CN)₆ respectively. The Ru(NH₃)₆²⁺ or Fe(CN)₆⁴⁻ then oxidised by the working electrode set at the appropriate potential. The current produced is proportional to the concentration of the target analyte. Various solutions containing a known amount of the analyte may be employed to calibrate the system and/or to produce standard curves allowing the concentration of the analyte to be determined in the sample being tested.

1. An electrochemical method for detecting and/or quantifying the amount of substrate in a sample, the method comprising
(i) contacting a redox enzyme, NAD⁺ or NADP⁺, a reductase and a redox active agent with said sample, wherein the redox enzyme is capable of oxidising or reducing the substrate using NAD⁺ or NADP⁺ as a cofactor; and
(ii) measuring the quantity of reduced redox active agent produced by electrochemical means.

2. A method according to claim 1, wherein the redox enzyme is a dehydrogenase.

3. A method according to claim 1, wherein the reductase is capable of accepting two electrons from NADH or NADPH.

4. A method according to claim 1, wherein the reductase is selected from putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b5 reductase, corn nitrate reductase, terpredoxin reductase and yeast, rat, rabbit and human NADPH cytochrome P450 reductase or a functional derivative of any thereof.

5. A method according to claim 1, wherein the redox active agent is Fe(CN)₆³⁻, Ru(NH₃)₆³⁺, or ferrocenium monocarboxylic acid (FMCA).

6. A method according to claim 1, wherein the reductase is specific for NADH.

7. A method according to claim 6, wherein the reductase is a putidaredoxin reductase.

8. A method according to claim 1, wherein the reductase is specific for NADPH.

9. A method according to claim 8, wherein the reductase is the flavin domain of P450_{BM-3} or is spinach ferrodoxin reductase.

10. A method according to claim 1, wherein the NAD⁺ or NADP⁺ is an NAD⁺ or NADP⁺ analog.

11. An electrochemical method for detecting and/or quantifying the amount of a redox enzyme in a sample, the method comprising
(i) contacting a substrate, NAD⁺ or NADP⁺, a reductase and a redox active agent with the sample, wherein the redox enzyme is capable of oxidising or reducing the substrate using NAD⁺ or NADP⁺ as a cofactor; and
(ii) measuring the quantity of reduced redox active agent produced by electrochemical means.

12. An electrochemical method for detecting and/or quantifying the amount of a target analyte in a sample, the method comprising
(i) reacting the target analyte with an enzyme to produce a substrate, or where the target analyte is an enzyme, reacting the target analyte with a precursor compound to produce a substrate,
(ii) contacting the thus produced substrate with a redox enzyme, NAD⁺ or NADP⁺, a reductase and a redox active agent, wherein the redox enzyme is capable of oxidising or reducing the substrate using NAD⁺ or NADP⁺ as a cofactor; and
(iii) measuring the quantity of reduced redox active agent produced by electrochemical means.

13. A method according to claim 12, wherein the reductase is a putidaredoxin reductase.

14. A method according to claim 12, wherein the NAD⁺ or NADP⁺ is an NAD⁺ or NADP⁺ analog.

15. An electrochemical detection sensor comprising an electrode system having a working electrode, a reference electrode and optionally a counter electrode and a buffered mixture of (i) a dehydrogenase enzyme for converting a target analyte to a product, (ii) NAD⁺ or NADP⁺, (iii) a NADH or NADPH reductase and (iv) a redox active agent.

16. A sensor according to claim 15, wherein the reductase is capable of accepting two electrons from NADH or NADPH.

17. A sensor according to claim 15, wherein the reductase is selected from putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductase or a functional derivative of any thereof.

18. A sensor according to claim 15, wherein the redox active agent is Fe(CN)₆³⁻, Ru(NH₃)₆³⁺, or ferrocenium monocarboxylic acid (FMCA).

19. A sensor according to claim 18, wherein the reductase is a putidaredoxin reductase.

20. A sensor according to claim 15, wherein the reductase is specific for NADPH.

21. A sensor according to claim 20, wherein the reductase is the flavin domain of P450_{BM-3} or is spinach ferrodoxin-reductase.

22. A sensor according to claim 15, wherein the NAD⁺ or NADP⁺ is an NAD⁺ or NADP⁺ analog.

23. A method of operating a sensor as defined in claim 15 which comprises
(i) contacting a liquid sample containing a substrate with the mixture of enzymes and redox agent such that the substrate is oxidised, producing NADH or NADPH which reduces the reductase, and the reductase in turn reduces the redox agent which is then oxidised at the working electrode; and
(ii) measuring the current produced.

24. An electrochemical cell comprising
(a) a sample holding means;
(b) a source of reductase;
(c) a redox active agent; and
(d) means for detecting and/or quantifying any current generated,
which electrochemical cell can be used to carry out a method as defined in claim 1.

25. An electrochemical cell comprising
(a) a sample holding means;
(b) a source of reductase;
(c) a redox active agent; and
(d) means for detecting and/or quantifying any current generated,
which electrochemical cell can be used to carry out a method as defined in claim 23.
